(19) United States
(12) Patent Application Publication
(10) Pub. No.: US 2015/0327585 A1
(43) Pub. Date: Nov. 19, 2015

(54) AQUEOUS CARBONATED BEVERAGE

(71) Applicant: TAISHO PHARMACEUTICAL CO., LTD., Tokyo (JP)

(72) Inventors: Takashi DOMOTO, Toshima-ku, Tokyo (JP); Marie YAMAJI, Toshima-ku, Tokyo (JP); Akane SAKATA, Toshima-ku, Tokyo (JP)

(73) Assignee: TAISHO PHARMACEUTICAL CO., LTD., Tokyo (JP)

(21) Appl. No.: 14/652,503

(22) PCT Filed: Dec. 12, 2013
(86) PCT No.: PCT/JP2013/083336

§ 371 Date: Jun. 16, 2015

(30) Foreign Application Priority Data
Dec. 25, 2012 (JP) 2012-280430

(51) Int. Cl. A23L 1/29; A23L 2/54; A61K 9/00; A23L 1/0524; A23L 1/0526; A61K 47/36; A23L 2/38; A23L 2/68

(52) U.S. Cl. CPC A23L 1/293; A23L 2/38; A23L 2/54; A23L 2/68; A23L 1/0524; A23L 1/0526; A61K 47/36; A61K 9/0095; A23V 2002/00

(57) ABSTRACT

An aqueous carbonated beverage comprises any one of:
0.01 w/v % or more of a LM pectin;
0.1 w/v % or more of alginic acid or a salt thereof; and 0.001 w/v % or more of gellan gum, wherein
the aqueous carbonated beverage has a pH of 3.5 to 7.0.

### TECHNICAL FIELD

The present invention relates to an aqueous carbonated beverage, and is utilizable in the fields of drugs, quasi drugs, foods, and so forth.

### BACKGROUND ART

Leading to metabolic syndrome, obesity is a serious social problem. Effective means for preventing obesity includes dieting: restricting amounts of meals taken. However, dieting cannot be continued for a long term due to hunger caused by the dieting per se in real circumstances. Hence, in order to eliminate hunger, a hunger reducing agent containing a flavor or a flavor compound as the main component (see PTL 1), a cereal food (see PTL 2), an edible phosphoprotein and a metal carbonate (see PTL 3), and the like have been provided.

As one of methods for alleviating such hunger, a method has been reported, which uses agar, for increasing the gel strength of the agar which is immersed in a gastric juice for a certain period (see PTL 4). Nevertheless, this method is hardly said to be highly practical because such hard gel has to be chewed and swallowed. Meanwhile, there have also been reports on a method which uses a gastric raft composition containing a gelling agent (see PTL 5) and a method which uses a gastric raft composition containing a gas producing material capable of producing a nontoxic gas when coming into contact with an aqueous acid (see PTL 6). These have a characteristic of floating in the stomach owing to the influence of the generated gas and are not easily digested by the peristaltic movement of the stomach. However, the compositions have to be taken in a large amount in the form of powder or tablet, and therefore have a problem of administration difficulty. Moreover, when these compositions are dissolved in water, the resulting solutions exhibit an alkalinity due to the influence of the gas generating substance. Hence, there are problems that the storability as a beverage is poor, and a beverage obtained by dissolving such a powder has a taste with a low palatability.

### CITATION LIST

### Patent Literatures

[PTL 1] Japanese Unexamined Patent Application Publication No. 2008-7427

[PTL 2] Japanese Unexamined Patent Application Publication No. 2007-53929

[PTL 3] Japanese Unexamined Patent Application Publication No. 2010-94085

[PTL 4] Japanese Unexamined Patent Application Publication No. 2008-110923

[PTL 5] International Application Japanese-Phase Publication No. 2009-530254

[PTL 6] International Application Japanese-Phase Publication No. 2005-507409

### SUMMARY OF INVENTION

### Technical Problem

An object of the present invention is to provide an aqueous carbonated beverage which is a normal aqueous carbonated beverage before drunken, but which turns into a gel by reaction with gastric acid in the stomach after drunken, so that the aqueous carbonated beverage has a characteristic of floating and remaining long in the stomach as a gel.

### Solution to Problem

The present inventors have conducted earnest study to achieve the above object. As a result, the inventors have found that an aqueous carbonated beverage comprising any one of 0.01 w/v % or more of a LM pectin, 0.1 w/v % or more of alginic acid or a salt thereof, and 0.001 w/v % or more of gellan gum, and having a pH set to 3.5 to 7.0, is a normal liquid (aqueous carbonated beverage) before drunken, but turns into a gel by reaction with gastric acid in the stomach after drunken, so that the aqueous carbonated beverage has a characteristic of floating and remaining long in the stomach as a gel. Moreover, since the form is a beverage, such an aqueous carbonated beverage is easily taken and can be designed in an acidic to neutral range. Thus, it is possible to design a beverage having a high storability and a taste with a high palatability.

Aspects of the present invention obtained from such findings are as follows.

(1) An aqueous carbonated beverage characterized by comprising any one of:

0.01 w/v % or more of a LM pectin;

0.1 w/v % or more of alginic acid or a salt thereof; and

0.001 w/v % or more of gellan gum, wherein

the aqueous carbonated beverage has a pH of 3.5 to 7.0.

(2) The aqueous carbonated beverage according to (1), where in the salt of alginic acid is any one of sodium alginate and potassium alginate.
(3) The aqueous carbonated beverage according to (1), wherein the gellan gum is deacylated gellan gum.

### Advantageous Effect of Invention

The present invention makes it possible to provide an aqueous carbonated beverage which has a characteristic of turning into a gel by reaction with gastric acid in the stomach after drunken, the gel floating and remaining long in the stomach.

### DESCRIPTION OF EMBODIMENTS

The term “pectin” refers to a water-soluble polysaccharide mainly containing α-1,4-linked polygalacturonic acid, and the compound is extracted from apples and Citrus. The pectin of the present invention may be derived from either apples or Citrus, but has to be a “LM pectin”: the ratio of galacturonic acid in the form of methyl ester is less than 50% (galacturonic acid is a constituent sugar of pectins and present in the free acid form or methyl ester form). Incidentally, a pectin with the methyl ester ratio of 50% or more is referred to as a HM pectin, and is not suitable in the present invention because such pectin does not turn into a gel in an acidic range.

An amount of the LM pectin contained (blended) in the beverage is 0.01 w/v % or more, preferably 0.025 to 5 w/v % in order that the gel strength and the administration easiness can be made excellent, and that the gel can be efficiently increased to a sufficient volume in the stomach. Note that, in the present invention, w/v % indicates a ratio of the mass (g) to a total volume of 100 ml.

The term “alginic acid” refers to a polysaccharide contained in seaweeds such as kelps (Laminariaceae) and wakame (Undaria pinnatifida), and is widely utilized as a thickening stabilizer. Alginic acid has a structure in which mannuronic acid and guluronic acid are linearly polymerized, and is commercially available in the form of a sodium salt, a potassium salt, a calcium salt, an ammonium salt, or the like. In the pre sent invention, either alginic acid or a salt thereof may be used. Any one of sodium alginate and potassium alginate is preferable from the viewpoint of solubility.

An amount of the alginic acid or salt thereof contained (blended) in the beverage is 0.1 w/v % or more, preferably 0.3 to 5 w/v % in order that the gel strength and the administration easiness can be made excellent, and that the gel can be efficiently increased to a sufficient volume in the stomach.

The term “gellan gum” refers to a polysaccharide produced by a microorganism (Sphingomonas elodea) extracellularly, and is widely utilized as a thickening stabilizer. Gellan gum is a linear heteropolysaccharide composed of repeating units each consisting of four sugars of glucose, glucuronic acid, glucose, and rhamnose, and has a carboxyl group derived from glucuronic acid. There are two types of gellan gum: deacylated gellan gum and native gellan gum, depending on the presence or absence of an acetyl group and a glyceryl group present in 1-3 linked glucose. In the present invention, any gellan gum may be used. Deacylated gellan gum is preferable from the viewpoint of gel strength.

An amount of the gellan gum contained (blended) in the beverage is 0.001 w/v % or more, preferably 0.0025 to 1 w/v % in order that the gel strength and the administration easiness can be made excellent, and that the gel can be efficiently increased to a sufficient volume in the stomach.

In the present invention, any one of the above-described LM pectin, alginic acid or salt thereof, and gellan gum may be used. From the viewpoint that the gel volume can be further increased in the stomach after drunken as a carbonated beverage, any one of the LM pectin and the alginic acid or salt thereof is preferably used, and the LM pectin is more preferably used.

The term “aqueous carbonated beverage” refers to one obtained by introducing carbon dioxide under pressure into water suitable for drinking, and then adding the resultant to a food, a food additive, or the like. The aqueous carbonated beverage preferably has a gas volume of 0.5 to 4.0. The term gas volume refers to a ratio of the volume of carbon dioxide to the volume of a liquid solvent in which the carbon dioxide is dissolved under the standard conditions (1 atm, 15.6° C.), provided that the volume of the liquid is taken as 1.

The aqueous carbonated beverage has a pH of 3.5 to 7.0 from the viewpoint that the aqueous carbonated beverage is a normal aqueous carbonated beverage before drunken but turns into a gel by reaction with gastric acid in the stomach after drunken. In order to keep the pH within the above-described range, a pH modifier may be blended as necessary. The pH modifier is preferably an organic acid. Particularly, citric acid, malic acid, and adipic acid are preferable from the viewpoints that the buffering capacity is high, and that the aqueous carbonated beverage hardly turns into a gel before drunken. An amount thereof in the beverage is 0.001 to 5 w/v %, more preferably 0.01 to 2 w/v % from the viewpoints of buffering capacity and administration easiness. Moreover, for the production within the above-described pH range, a preservative may be blended as necessary. Particularly, the preservative is preferably benzoic acid and benzoic acid salts such as sodium benzoate, and an amount thereof in the beverage is 0.001 to 0.5 w/v %, more preferably 0.005 to 0.1 w/v % from the viewpoints of storability and administration easiness.

The aqueous carbonated beverage can be produced according to a conventionally known method. For example, each component is added to and dissolved in water by mixing. Thereby, a raw solution of the beverage is prepared. Then, the solution is subjected to pH adjustment and sterilization by heating as necessary. After cooling, carbon dioxide gas is incorporated thereinto (carbonation) in such a manner that the gas pressure is within a predetermined range. The resulting solution is filled into a container, followed by a sterilization step. Thus, the aqueous carbonated beverage can be produced.

Note that although the method for producing the carbonated beverage includes a pre-mix method and a post-mix method, any one of the methods may be used in the present invention.

Further, as other ingredients, a vitamin, a mineral, an amino acid and a salt thereof, a crude drug, a crude drug extract, caffeine, royal jelly, a dextrin, or the like may be blended in the aqueous carbonated beverage as appropriate, as long as the effect of the present invention is not impaired. Furthermore, as necessary, an additive such as an antioxidant, a colorant, a flavor, a taste masking agent, a preservative, or a sweetener may be blended as appropriate, as long as the effect of the present invention is not impaired.

### EXAMPLES

Hereinafter, the present invention will be described in further detail by illustrating Examples, Comparative Examples, and Test Example.

### Examples and Comparative Examples

Citric acid monohydrate and sodium benzoate were added to and dissolved in purified water. The pH was adjusted with hydrochloric acid and an aqueous solution of sodium hydroxide. Each of a LM pectin, a HM pectin, sodium alginate, gellan gum, carrageenan, or xanthan gum was dissolved in the solution. Purified water was further added to the solution to make the total amount 25 ml. The pH was finely adjusted with an aqueous solution of sodium hydroxide. Each test solution was filled into a glass bottle and sterilized, and 75 ml of carbonated water or purified water was added thereto immediately before the evaluation. Thus, aqueous beverages of Examples 1 to 8, 10, 11, 13 to 16, 18, and 19 and of Comparative Examples 1 to 6 were obtained.

In Examples 9, 12, and 17, citric acid monohydrate and sodium benzoate were added to purified water, and each of a LM pectin, sodium alginate, or gellan gum was dissolved therein. Purified water was further added to the solution to make the total amount 25 ml. After carbonated water was added thereto, the pH was adjusted with hydrochloric acid and a solution of sodium hydroxide. The total amount was made to be 100 ml.

Each of the aqueous beverages obtained in Examples 1 to 19 was an aqueous carbonated beverage having a gas volume within a range of 0.5 to 4.0 and a total amount of 100 ml. Tables 1 to 5 show the composition and the pH of each aqueous beverage.

TABLE 1

| | | | | | Comparative | Comparative | Comparative | Comparative |
|---|---|---|---|---|---|---|---|---|
| Component name | Unit | Example 1 | Example 2 | Example 3 | Example 1 | Example 2 | Example 3 | Example 4 |
| LM pectin | mg | 500 | — | — | 500 | — | — | — |
| HM pectin | mg | — | — | — | — | 500 | — | — |
| sodium alginate | mg | — | 500 | — | — | — | — | — |
| gellan gum | mg | — | — | 50 | — | — | — | — |
| carrageenan | mg | — | — | — | — | — | 50 | — |
| xanthan gum | mg | — | — | — | — | — | — | 50 |
| citric acid | mg | 200 | 200 | 50 | 200 | 200 | 50 | 200 |
| monohydrate | | | | | | | | |
| sodium benzoate | mg | 50 | 50 | 50 | 50 | 50 | 50 | 50 |
| hydrochloric acid | mg | adequate | adequate | adequate | adequate | adequate | adequate | adequate |
| | | amount | amount | amount | amount | amount | amount | amount |
| aqueous solution of | mg | adequate | adequate | adequate | adequate | adequate | adequate | adequate |
| sodium hydroxide | | amount | amount | amount | amount | amount | amount | amount |
| purified water, total | ml | — | — | — | 100 | — | — | — |
| carbonated water, | ml | 100 | 100 | 100 | — | 100 | 100 | 100 |
| total | | | | | | | | |

| | | | | | | | |
|---|---|---|---|---|---|---|---|
| pH | 4.1 | 4.3 | 4.4 | 4.1 | 4.1 | 4.1 | 4.1 |

TABLE 2

| Component name | Unit | Example 1 | Example 4 | Example 5 | Example 6 | Example 7 | Example 8 | Example 9 |
|---|---|---|---|---|---|---|---|---|
| LM pectin | mg | 500 | 25 | 50 | 100 | 200 | 500 | 500 |
| citric acid | mg | 200 | 200 | 200 | 200 | 200 | 200 | 200 |
| monohydrate | | | | | | | | |
| sodium benzoate | mg | 50 | 50 | 50 | 50 | 50 | 50 | 50 |
| hydrochloric acid | mg | adequate | adequate | adequate | adequate | adequate | adequate | adequate |
| | | amount | amount | amount | amount | amount | amount | amount |
| aqueous solution | mg | adequate | adequate | adequate | adequate | adequate | adequate | adequate |
| of sodium | | amount | amount | amount | amount | amount | amount | amount |
| hydroxide | | | | | | | | |
| carbonated water, | ml | 100 | 100 | 100 | 100 | 100 | 100 | 100 |
| total | | | | | | | | |

| | | | | | | | |
|---|---|---|---|---|---|---|---|
| pH | 4.1 | 4.1 | 4.1 | 4.1 | 4.1 | 3.6 | 6.8 |

TABLE 3

| | | Comparative | | | | |
|---|---|---|---|---|---|---|
| Component name | Unit | Example 5 | Example 2 | Example 10 | Example 11 | Example 12 |
| sodium alginate | mg | 500 | 500 | 300 | 500 | 1000 |
| citric acid | mg | 200 | 200 | 200 | 200 | 200 |
| monohydrate | | | | | | |
| sodium benzoate | mg | 50 | 50 | 50 | 50 | 50 |
| hydrochloric acid | mg | adequate | adequate | adequate | adequate | adequate |
| | | amount | amount | amount | amount | amount |
| aqueous solution | mg | adequate | adequate | adequate | adequate | adequate |
| of sodium | | amount | amount | amount | amount | amount |
| hydroxide | | | | | | |
| purified water, | ml | 100 | — | — | — | — |
| total | | | | | | |
| carbonated water, | ml | — | 100 | 100 | 100 | 100 |
| total | | | | | | |

| | | | | | |
|---|---|---|---|---|---|
| pH | 4.3 | 4.3 | 4.3 | 3.9 | 6.7 |

TABLE 4

| | | Comparative | | | | | | |
|---|---|---|---|---|---|---|---|---|
| Component name | Unit | Example 6 | Example 3 | Example 13 | Example 14 | Example 15 | Example 16 | Example 17 |
| gellan gum | mg | 50 | 50 | 2.5 | 5 | 10 | 50 | 50 |
| citric acid | mg | 50 | 50 | 50 | 50 | 50 | 50 | 50 |
| monohydrate | | | | | | | | |
| sodium benzoate | mg | 50 | 50 | 50 | 50 | 50 | 50 | 50 |
| hydrochloric acid | mg | adequate | adequate | adequate | adequate | adequate | adequate | adequate |
| | | amount | amount | amount | amount | amount | amount | amount |
| aqueous solution of | mg | adequate | adequate | adequate | adequate | adequate | adequate | adequate |
| sodium hydroxide | | amount | amount | amount | amount | amount | amount | amount |
| purified water, | ml | 100 | — | — | — | — | — | — |
| total | | | | | | | | |
| carbonated water, | ml | — | 100 | 100 | 100 | 100 | 100 | 100 |
| total | | | | | | | | |

| | | | | | | | |
|---|---|---|---|---|---|---|---|
| pH | 4.6 | 4.4 | 4.5 | 4.5 | 4.5 | 4.3 | 6.7 |

TABLE 5

| | | Example | Example |
|---|---|---|---|
| Component name | Unit | 18 | 19 |
| LM pectin | mg | 2000 | 2000 |
| citric acid monohydrate | mg | 200 | 200 |
| sodium benzoate | mg | 50 | 50 |
| hydrochloric acid | mg | adequate amount | adequate amount |
| aqueous solution of sodium | mg | adequate amount | adequate amount |
| hydroxide | | | |
| carbonated water, total | ml | 100 | 100 |

| | | |
|---|---|---|
| pH | 3.9 | 4.2 |

### Test Example

### State and Volume at the Time of Dripping onto Artificial Gastric Juice

On 100 ml of a Japanese Pharmacopoeia first fluid (first solution for Disintegration Test in Japanese Pharmacopoeia) at 37° C. as an artificial gastric juice, 100 ml of each of the aqueous beverages of Examples and Comparative Examples cooled with ice water was dripped. After the incubation at 37° C. for 10 minutes, the state was observed, and the total volume was measured. Tables 6 to 10 show the result. Note that, in the tables, “floating” indicates a state where the gelled aqueous beverage was floating on the Japanese Pharmacopoeia first fluid.

TABLE 6

| | | | | | Comparative | Comparative | Comparative | Comparative |
|---|---|---|---|---|---|---|---|---|
| | Unit | Example 1 | Example 2 | Example 3 | Example 1 | Example 2 | Example 3 | Example 4 |
| State before loading | | liquid | liquid | liquid | liquid | liquid | liquid | liquid |
| onto Japanese | | | | | | | | |
| Pharmacopoeia first | | | | | | | | |
| fluid | | | | | | | | |
| State after loading | | gel | gel | gel | gel | liquid | liquid | liquid |
| onto Japanese | | | | | | | | |
| Pharmacopoeia first | | | | | | | | |
| fluid | | | | | | | | |
| State of gel after | | floating | floating | floating | no floating | | | |
| loading | | | | | | | | |
| Volume after | ml | 272 | 236 | 216 | 203 | 200 | 200 | 199 |
| incubation | | | | | | | | |

TABLE 7

| | Unit | Example 1 | Example 4 | Example 5 | Example 6 | Example 7 | Example 8 | Example 9 |
|---|---|---|---|---|---|---|---|---|
| State before loading | | liquid | liquid | liquid | liquid | liquid | liquid | liquid |
| onto Japanese | | | | | | | | |
| Pharmacopoeia first | | | | | | | | |
| fluid | | | | | | | | |
| State after loading | | gel | gel | gel | gel | gel | gel | gel |
| onto Japanese | | | | | | | | |
| Pharmacopoeia first | | | | | | | | |
| fluid | | | | | | | | |
| State of gel after | | floating | floating | floating | floating | floating | floating | floating |
| loading | | | | | | | | |
| Volume after | ml | 272 | 206 | 212 | 221 | 236 | 246 | 242 |
| incubation | | | | | | | | |

TABLE 8

| | | Comparative | | | | |
|---|---|---|---|---|---|---|
| | Unit | Example 5 | Example 2 | Example 10 | Example 11 | Example 12 |
| State before loading | | liquid | liquid | liquid | liquid | liquid |
| onto Japanese | | | | | | |
| Pharmacopoeia first | | | | | | |
| fluid | | | | | | |
| State after loading | | gel | gel | gel | gel | gel |
| onto Japanese | | | | | | |
| Pharmacopoeia first | | | | | | |
| fluid | | | | | | |
| State of gel after | | no floating | floating | floating | floating | floating |
| loading | | | | | | |
| Volume after | ml | 200 | 236 | 204 | 228 | 204 |
| incubation | | | | | | |

TABLE 9

| | | Comparative | | | | | | |
|---|---|---|---|---|---|---|---|---|
| | Unit | Example 6 | Example 3 | Example 13 | Example 14 | Example 15 | Example 16 | Example 17 |
| State before loading | | liquid | liquid | liquid | liquid | liquid | liquid | liquid |
| onto Japanese | | | | | | | | |
| Pharmacopoeia first | | | | | | | | |
| fluid | | | | | | | | |
| State after loading | | gel | gel | gel | gel | gel | gel | gel |
| onto Japanese | | | | | | | | |
| Pharmacopoeia first | | | | | | | | |
| fluid | | | | | | | | |
| State of gel after | | no floating | floating | floating | floating | floating | floating | floating |
| loading | | | | | | | | |
| Volume after | ml | 200 | 216 | 204 | 208 | 206 | 216 | 228 |
| incubation | | | | | | | | |

TABLE 10

| | | Example | Example |
|---|---|---|---|
| | Unit | 18 | 19 |
| State before loading onto Japanese | | liquid | liquid |
| Pharmacopoeia first fluid | | | |
| State after loading onto Japanese | | gel | gel |
| Pharmacopoeia first fluid | | | |
| State of gel after loading | | floating | floating |
| volume after incubation | ml | 212 | 236 |

Unlike the simple aqueous beverage comprising the LM pectin as in Comparative Example 1, the aqueous carbonated beverage comprising the LM pectin as in Example 1 expanded as bubbles of carbon dioxide gas were formed inside the gel. As a result, the total volume was increased, the relative density was decreased, and the aqueous carbonated beverage floated. This was the same for the aqueous carbonated beverages comprising sodium alginate or gellan gum (Examples 2 and 3, compared with Comparative Examples 5 and 6).

Meanwhile, as for the aqueous beverages of Comparative Examples 2 to 4 respectively comprising the HM pectin, carrageenan, and xanthan gum, no gelation was observed after the dripping onto the Japanese Pharmacopoeia first fluid.

Moreover, the gelation and floating were observed when the amount of the LM pectin blended was 0.025 w/v % or more as illustrated in Examples 4 to 7, and when the pH was within the range of 3.6 to 6.8 as illustrated in Examples 8 and 9. As to sodium alginate, the gelation and floating were observed when the blended amount was 0.3 w/v % or more as illustrated in Example 10, and when the pH was within the range of 3.9 to 6.7 as illustrated in Examples 11 and 12. As to the gellan gum, the gelation and floating were observed when the blended amount was 0.0025 w/v % or more as illustrated in Examples 13 to 15, and when the pH was within the range of 4.3 to 6.7 as illustrated in Examples 16 and 17.

As illustrated in Examples 18 and 19, the gelation and floating were observed even in the low-pH solutions comprising a high concentration of the LM pectin.

### INDUSTRIAL APPLICABILITY

The present invention makes it possible to provide an aqueous carbonated beverage which is a normal aqueous carbonated beverage before drunken, but which turns into a gel by reaction with gastric acid in the stomach after drunken, so that the aqueous carbonated beverage has a characteristic of floating and remaining long in the stomach as a gel. Therefore, by providing the present invention as drugs, quasi drugs, and foods which are directed to dieting for preventing obesity, these industries are expected to grow.

1. An aqueous carbonated beverage comprising any one of:
0.01 w/v % or more of a LM pectin;
0.1 w/v % or more of alginic acid or a salt thereof; and
0.001 w/v % or more of gellan gum, wherein
the aqueous carbonated beverage has a pH of 3.5 to 7.0.

2. The aqueous carbonated beverage according to claim 1, wherein the salt of alginic acid is any one of sodium alginate and potassium alginate.

3. The aqueous carbonated beverage according to claim 1, wherein the gellan gum is deacylated gellan gum.
